(19) United States
(12) Patent Application Publication
(10) Pub. No.: US 2024/0174624 A1
(43) Pub. Date: May 30, 2024

(54) CHIRAL N-(1,3,4-OXADIAZOLE-2-YL)PHENYL CARBOXYLIC ACID AMIDES AND THEIR USE AS HERBICIDES

(71) Applicant: Bayer Aktiengesellschaft, Leverkusen (DE)

(72) Inventors: Arnim KOEHN, Leverkusen (DE); Christian WALDRAFF, Leverkusen (DE); Hartmut AHRENS, Leverkusen (DE); Ralf BRAUN, Leverkusen (DE); Harald JAKOBI, Leverkusen (DE); Christopher Hugh ROSINGER, Leverkusen (DE); Elmar GATZWEILER, Leverkusen (DE); Jan DITTGEN, Leverkusen (DE); Birgit BOLLENBACH-WAHL, Leverkusen (DE); Elisabeth ASMUS, Leverkusen (DE)

(21) Appl. No.: 18/549,497

(22) PCT Filed: Mar. 9, 2022
(86) PCT No.: PCT/EP2022/056017

§ 371 Date: Sep. 7, 2023

(30) Foreign Application Priority Data
Mar. 12, 2021 (EP) 21162218.8

(51) Int. Cl. C07D 271/113; A01N 43/82; A01P 13/00

(52) U.S. Cl. CPC C07D 271/113; A01N 43/82; A01P 13/00

(57) ABSTRACT

Chiral N-(1,3,4-oxadiazole-2-yl)phenyl carboxylic acid amides of the general formula (I) and the use thereof as herbicides are described.

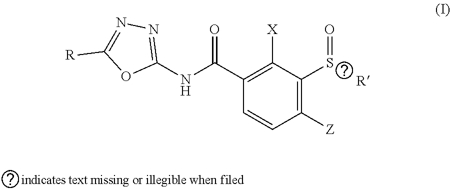

In this formula (I), X, Z, R and R′ are radicals such as alkyl, cycloalkyl and halogen.

The invention relates to the technical field of the herbicides, especially that of the herbicides for selective control of weeds and weed grasses in crops of useful plants.

WO 2012/126932 A1 and WO 2018/202535 A1 describe herbicidally active benzoylamides that differ from one another essentially by the nature of the N-bonded heterocyclic substituent and the substituents on the phenyl ring. Among the substances disclosed in both documents are (1,3,4-oxadiazole-2-yl)phenyl carboxamides that bear different alkylsulfinyl radicals in the 3 position of the phenyl ring, without specifying the stereochemistry of these chiral alkylsulfinyl radicals.

However, the benzoylamides known from the above-cited documents often have an unfavourable profile with regard to their biological properties, such as herbicidal action, tolerance by crop plants, toxicological and ecotoxicological properties.

It is an object of the present invention to provide alternative herbicidally active ingredients. This object is achieved by the (1,3,4-oxadiazole-2-yl)phenyl carboxamides of the invention that are described hereinafter, which bear a chiral alkylsulfinyl radical with a stereochemically defined configuration in the 3 position of the phenyl ring.

The present invention provides N-(1,3,4-oxadiazole-2-yl)phenyl carboxamides or salts thereof having the absolute configuration specified in formula (I)

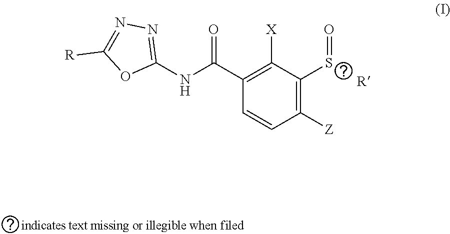

in which the substituents are defined as follows:
R is hydrogen or methyl,
X is Cl or methyl,
R′ is methyl or c-Pr,
Z is CF₃ or CHF₂.

Compounds of the invention are those of the general formula (I) which, according to the Cahn-Ingold-Prelog rules, are in the S configuration, provided that R′ has a lower priority than the phenyl ring. This is true, for example, of compounds of the general formula (I) in which R′ is methyl or cyclopropyl. Further compounds of the invention are those of the general formula (I) which, according to the Cahn-Ingold-Prelog rules, are in the R configuration, provided that R′ has a higher priority than the phenyl ring.

Preference is given to compounds I-1 to I-16:

| No. | R | X | R′ | Z |
|---|---|---|---|---|
| I-1 | H | Cl | Me | CF₃ |
| I-2 | H | Cl | Me | CHF₂ |
| I-3 | H | Me | Me | CF₃ |
| I-4 | Me | Cl | c-Pr | CF₃ |
| I-5 | Me | Cl | Me | CF₃ |
| I-6 | Me | Cl | Me | CHF₂ |
| I-7 | H | Me | Me | CHF₂ |
| I-8 | H | Me | c-Pr | CF₃ |
| I-9 | H | Me | c-Pr | CHF₂ |
| I-10 | H | Cl | c-Pr | CF₃ |
| I-11 | H | Cl | c-Pr | CHF₂ |
| I-12 | Me | Cl | c-Pr | CHF₂ |
| I-13 | Me | Me | Me | CF₃ |
| I-14 | Me | Me | Me | CHF₂ |
| I-15 | Me | Me | c-Pr | CF₃ |
| I-16 | Me | Me | c-Pr | CHF₂ |

Particular preference is given to compounds I-1 to I-5:

| No. | R | X | R′ | Z |
|---|---|---|---|---|
| I-1 | H | Cl | Me | CF₃ |
| I-2 | H | Cl | Me | CHF₂ |
| I-3 | H | Me | Me | CF₃ |
| I-4 | Me | Cl | c-Pr | CF₃ |
| I-5 | Me | Cl | Me | CF₃ |

Very particular preference is given to compounds I-4 and I-5:

| No. | R | X | R′ | Z |
|---|---|---|---|---|
| I-4 | Me | Cl | c-Pr | CF₃ |
| I-5 | Me | Cl | Me | CF₃ |

In all the formulae specified hereinafter, the substituents and symbols have the same meaning as described in formula (I), unless defined differently.

Inventive compounds of the general formula (I) can be prepared, for example, by chromatographic enantiomer separation of the racemic N-(1,3,4-oxadiazole-2-yl)phenyl carboxamides (Ia), as specified in Scheme 1.

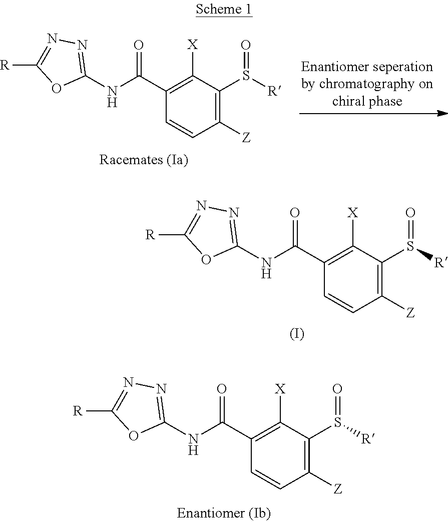

The racemic N-(1,3,4-oxadiazole-2-yl)phenyl carboxamides (Ia) are known in principle and can be prepared, for example, by the methods described in WO 2012/126932 or WO 2018/202535.

The inventive compounds of the formula (I) have excellent herbicidal activity against a broad spectrum of economically important mono- and dicotyledonous annual harmful plants.

The present invention therefore also provides a method for controlling unwanted plants or for regulating the growth of plants, preferably in plant crops, in which one or more compound(s) of the invention is/are applied to the plants (for example harmful plants such as monocotyledonous or dicotyledonous weeds or unwanted crop plants), the seed (for example grains, seeds or vegetative propagules such as tubers or shoot parts with buds) or the area on which the plants grow (for example the area under cultivation). The compounds of the invention can be deployed, for example, prior to sowing (if appropriate also by incorporation into the soil), prior to emergence or after emergence. Specific examples of some representatives of the monocotyledonous and dicotyledonous weed flora which can be controlled by the compounds of the invention are as follows, though the enumeration is not intended to impose a restriction to particular species.

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous weeds of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

When the compounds of the invention are applied to the soil surface before germination, either the weed seedlings are prevented completely from emerging or the weeds grow until they have reached the cotyledon stage, but then stop growing.

If the active ingredients are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner.

The compounds of the invention can be selective in crops of useful plants and can also be employed as non-selective herbicides.

By virtue of their herbicidal and plant growth regulatory properties, the active ingredients can also be used to control harmful plants in crops of genetically modified plants which are known or are yet to be developed. In general, the transgenic plants are characterized by particular advantageous properties, for example by resistances to certain active ingredients used in the agrochemical industry, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material. Further particular properties lie in tolerance or resistance to abiotic stress factors, for example heat, cold, drought, salinity and ultraviolet radiation.

Preference is given to using the inventive compounds of the formula (I) in economically important transgenic crops of useful and ornamental plants.

The compounds of the formula (I) can be used as herbicides in crops of useful plants which are resistant, or have been made resistant by genetic engineering, to the phytotoxic effects of the herbicides.

Conventional ways of producing novel plants which have modified properties in comparison to existing plants consist, for example, in traditional cultivation methods and the generation of mutants. Alternatively, novel plants with altered properties can be generated with the aid of recombinant methods (see, for example, EP 0221044, EP 0131624). What have been described are, for example, several cases of genetic modifications of crop plants for the purpose of modifying the starch synthesized in the plants (e.g. WO 92/011376 A, WO 92/014827 A, WO 91/019806 A), transgenic crop plants which are resistant to certain herbicides of the glufosinate type (cf., for example, EP 0242236 A, EP 0242246 A) or of the glyphosate type (WO 92/000377 A) or of the sulfonylurea type (EP 0257993 A, U.S. Pat. No. 5,013,659) or to combinations or mixtures of these herbicides through “gene stacking”, such as transgenic crop plants, for example corn or soya with the trade name or the designation Optimum™ GAT™ (Glyphosate ALS Tolerant).
transgenic crop plants, for example cotton, capable of producing Bacillus thuringiensis toxins (Bt toxins), which make the plants resistant to particular pests (EP 0142924 A, EP 0193259 A),
transgenic crop plants having a modified fatty acid composition (WO 91/013972 A),
genetically modified crop plants having novel constituents or secondary metabolites, for example novel phytoalexins, which cause an increase in disease resistance (EP 0309862 A, EP 0464461 A),
genetically modified plants having reduced photorespiration, which have higher yields and higher stress tolerance (EP 0305398 A),
transgenic crop plants which produce pharmaceutically or diagnostically important proteins (“molecular pharming”),
transgenic crop plants which feature higher yields or better quality,
transgenic crop plants which are distinguished by a combination, for example of the abovementioned novel properties (“gene stacking”).

Numerous molecular biology techniques which can be used to produce novel transgenic plants with modified properties are known in principle; see, for example, I. Potrykus and G. Spangenberg (eds), Gene Transfer to Plants, Springer Lab Manual (1995), Springer Verlag Berlin, Heidelberg or Christou, “Trends in Plant Science” 1 (1996) 423-431).

For such genetic manipulations, nucleic acid molecules which allow mutagenesis or sequence alteration by recombination of DNA sequences can be introduced into plasmids. With the aid of standard methods, it is possible, for example, to undertake base exchanges, remove part sequences or add natural or synthetic sequences. For the connection of the DNA fragments to one another, it is possible to add adapters or linkers to the fragments; see, for example, Sambrook et al., 1989, Molecular Cloning, A Laboratory Manual, 2nd ed., Cold Spring Harbor Laboratory Press, Cold Spring Harbor, NY; or Winnacker “Gene and Klone” [Genes and Clones], VCH Weinheim, 2nd edition, 1996.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product. To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Such sequences are known to those skilled in the art (see, for example, Braun et al., EMBO J. 11 (1992), 3219-3227; Wolter et al., Proc. Natl. Acad. Sci. USA 85 (1988), 846-850; Sonnewald et al., Plant J. 1 (1991), 95-106). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants. Obtainable in this way are transgenic plants having properties altered by overexpression, suppression or inhibition of homologous (=natural) genes or gene sequences or expression of heterologous (=foreign) genes or gene sequences.

The compounds (I) of the invention can be used with preference in transgenic crops which are resistant to growth regulators, for example 2,4-D, dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, the glyphosates, glufosinates or benzoylisoxazoles and analogous active ingredients, or to any desired combinations of these active ingredients.

The compounds of the invention can be used with particular preference in transgenic crop plants which are resistant to a combination of glyphosates and glufosinates, glyphosates and sulfonylureas or imidazolinones. Most preferably, the compounds of the invention can be used in transgenic crop plants such as corn or soya with the trade name or the designation Optimum™ GAT™ (glyphosate ALS tolerant), for example.

When the active ingredients of the invention are employed in transgenic crops, not only do the effects towards harmful plants observed in other crops occur, but frequently also effects which are specific to the application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the inventive compounds of the formula (I) as herbicides for controlling harmful plants in transgenic crop plants.

The compounds of the invention can be applied in the form of wettable powders, emulsifiable concentrates, sprayable solutions, dusting products or granules in the customary formulations. The invention therefore also provides herbicidal and plant-growth-regulating compositions which comprise the compounds of the invention.

The compounds of the invention can be formulated in various ways, according to the biological and/or physicochemical parameters required. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes. These individual formulation types are known in principle and are described, for example, in: Winnacker-Küchler, “Chemische Technologie” [Chemical Technology], Volume 7, C. Hanser Verlag Munich, 4th Ed. 1986, Wade van Valkenburg, “Pesticide Formulations”, Marcel Dekker, N.Y., 1973, K. Martens, “Spray Drying” Handbook, 3rd Ed. 1979, G. Goodwin Ltd. London.

The necessary formulation auxiliaries, such as inert materials, surfactants, solvents and further additives, are likewise known and are described, for example, in: Watkins, “Handbook of Insecticide Dust Diluents and Carriers”, 2nd ed., Darland Books, Caldwell N.J., H.v. Olphen, “Introduction to Clay Colloid Chemistry”, 2nd ed., J. Wiley & Sons, N.Y., C. Marsden, “Solvents Guide”, 2nd ed., Interscience, N.Y. 1963, McCutcheon's “Detergents and Emulsifiers Annual”, MC Publ. Corp., Ridgewood N.J., Sisley and Wood, “Encyclopedia of Surface Active Agents”, Chem. Publ. Co. Inc., N.Y. 1964, Schönfeldt, “Grenzflächenaktive Äthylenoxidaddukte” [Interface-active Ethylene Oxide Adducts], Wiss. Verlagsgesell., Stuttgart 1976, Winnacker-Küchler, “Chemische Technologie”, Volume 7, C. Hanser Verlag Munich, 4th ed. 1986.

On the basis of these formulations, it is also possible to produce combinations with other active ingredients, for example insecticides, acaricides, herbicides, fungicides, and also with safeners, fertilizers and/or growth regulators, for example in the form of a finished formulation or as a tank mix.

Wettable powders are preparations uniformly dispersible in water which, in addition to the active ingredient and apart from a diluent or inert substance, also comprise surfactants of ionic and/or nonionic type (wetting agent, dispersant), e.g. polyethoxylated alkylphenols, polyethoxylated fatty alcohols, polyethoxylated fatty amines, fatty alcohol polyglycolethersulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the active herbicidal ingredients are finely ground, for example in customary apparatuses such as hammer mills, blower mills and air-jet mills, and simultaneously or subsequently mixed with the formulation auxiliaries.

Emulsifiable concentrates are produced by dissolving the active ingredient in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonate salts such as calcium dodecylbenzenesulfonate, or nonionic emulsifiers such as fatty acid polyglycol esters, alkylaryl polyglycol ethers, fatty alcohol polyglycol ethers, propylene oxide/ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active ingredient with finely distributed solids, for example talc, natural clays, such as kaolin, bentonite and pyrophyllite, or diatomaceous earth.

Suspension concentrates may be water- or oil-based. They may be produced, for example, by wet-grinding by means of commercial bead mills and optional addition of surfactants as already listed above, for example, for the other formulation types.

Emulsions, for example oil-in-water emulsions (EW), can be produced, for example, by means of stirrers, colloid mills and/or static mixers using aqueous organic solvents and optionally surfactants as already listed above, for example, for the other formulation types.

Granules can be produced either by spraying the active ingredient onto granular inert material capable of adsorption or by applying active ingredient concentrates to the surface of carrier substances, such as sand, kaolinites or granular inert material, by means of adhesives, for example polyvinyl alcohol, sodium polyacrylate or else mineral oils. Suitable active ingredients can also be granulated in the manner customary for the production of fertilizer granules—if desired as a mixture with fertilizers.

Water-dispersible granules are produced generally by the customary processes such as spray-drying, fluidized-bed granulation, pan granulation, mixing with high-speed mixers and extrusion without solid inert material.

For the production of pan granules, fluidized bed granules, extruder granules and spray granules, see, for example, processes in “Spray-Drying Handbook” 3rd ed. 1979, G. Goodwin Ltd., London, J. E. Browning, “Agglomeration”, Chemical and Engineering 1967, pages 147 ff.; “Perry's Chemical Engineer's Handbook”, 5th Ed., McGraw-Hill, New York 1973, pp. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical preparations contain generally 0.1% to 99% by weight, especially 0.1% to 95% by weight, of compounds of the invention. In wettable powders, the active ingredient concentration is, for example, about 10% to 90% by weight, the remainder to 100% by weight consisting of customary formulation constituents. In emulsifiable concentrates, the active ingredient concentration may be about 1% to 90% and preferably 5% to 80% by weight. Formulations in the form of dusts comprise 1% to 30% by weight of active ingredient, preferably usually 5% to 20% by weight of active ingredient; sprayable solutions contain about 0.05% to 80% by weight, preferably 2% to 50% by weight of active ingredient. In the case of water-dispersible granules, the active ingredient content depends partially on whether the active ingredient is in liquid or solid form and on which granulation auxiliaries, fillers, etc., are used. In the water-dispersible granules, the content of active ingredient is, for example, between 1% and 95% by weight, preferably between 10% and 80% by weight.

In addition, the active ingredient formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity.

On the basis of these formulations, it is also possible to produce combinations with other pesticidally active substances, for example insecticides, acaricides, herbicides, fungicides, and also with safeners, fertilizers and/or growth regulators, for example in the form of a finished formulation or as a tank mix.

For application, the formulations in commercial form are, if appropriate, diluted in a customary manner, for example in the case of wettable powders, emulsifiable concentrates, dispersions and water-dispersible granules with water. Dust-type preparations, granules for soil application or granules for scattering and sprayable solutions are not normally diluted further with other inert substances prior to application.

The required application rate of the compounds of the formula (I) varies with the external conditions, including, inter alia, temperature, humidity and the type of herbicide used. It can vary within wide limits, for example between 0.001 and 10.0 kg/ha or more of active substance, but it is preferably between 0.005 and 5 kg/ha, more preferably in the range of from 0.01 to 1.5 kg/ha, more preferably in the range of from 0.05 to 1 kg/ha. This applies both to pre-emergence and to post-emergence application.

A carrier is a natural or synthetic, organic or inorganic substance with which the active ingredients are mixed or combined for better applicability, in particular for application to plants or plant parts or seed. The carrier, which may be solid or liquid, is generally inert and should be suitable for use in agriculture.

Useful solid or liquid carriers include: for example ammonium salts and natural rock dusts, such as kaolins, clays, talc, chalk, quartz, attapulgite, montmorillonite or diatomaceous earth, and synthetic rock dusts, such as finely divided silica, alumina and natural or synthetic silicates, resins, waxes, solid fertilizers, water, alcohols, especially butanol, organic solvents, mineral and vegetable oils, and derivatives thereof. It is likewise possible to use mixtures of such carriers. Useful solid carriers for granules include: for example crushed and fractionated natural rocks such as calcite, marble, pumice, sepiolite, dolomite, and synthetic granules of inorganic and organic meals, and also granules of organic material such as sawdust, coconut shells, corn cobs and tobacco stalks.

Suitable liquefied gaseous extenders or carriers are liquids which are gaseous at standard temperature and under atmospheric pressure, for example aerosol propellants such as halogenated hydrocarbons, or else butane, propane, nitrogen and carbon dioxide.

In the formulations, it is possible to use tackifiers such as carboxymethylcellulose, natural and synthetic polymers in the form of powders, granules or latices, such as gum arabic, polyvinyl alcohol and polyvinyl acetate, or else natural phospholipids such as cephalins and lecithins, and synthetic phospholipids. Further additives may be mineral and vegetable oils.

When the extender used is water, it is also possible to use, for example, organic solvents as auxiliary solvents. Useful liquid solvents are essentially: aromatics such as xylene, toluene or alkylnaphthalenes, chlorinated aromatics or chlorinated aliphatic hydrocarbons such as chlorobenzenes, chloroethylenes or dichloromethane, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulfoxide, and also water.

The compositions of the invention may additionally comprise further components, for example surfactants. Useful surfactants are emulsifiers and/or foam formers, dispersants or wetting agents having ionic or nonionic properties, or mixtures of these surfactants. Examples thereof are salts of polyacrylic acid, salts of lignosulfonic acid, salts of phenolsulfonic acid or naphthalenesulfonic acid, polycondensates of ethylene oxide with fatty alcohols or with fatty acids or with fatty amines, substituted phenols (preferably alkylphenols or arylphenols), salts of sulfosuccinic esters, taurine derivatives (preferably alkyl taurates), phosphoric esters of polyethoxylated alcohols or phenols, fatty acid esters of polyols, and derivatives of the compounds containing sulfates, sulfonates and phosphates, for example alkylaryl polyglycol ethers, alkylsulfonates, alkyl sulfates, arylsulfonates, protein hydrolyzates, lignosulfite waste liquors and methylcellulose. The presence of a surfactant is necessary if one of the active ingredients and/or one of the inert carriers is insoluble in water and when application is effected in water. The proportion of surfactants is between 5 and 40 percent by weight of the inventive composition. It is possible to use dyes such as inorganic pigments, for example iron oxide, titanium oxide and Prussian Blue, and organic dyes such as alizarin dyes, azo dyes and metal phthalocyanine dyes, and trace nutrients such as salts of iron, manganese, boron, copper, cobalt, molybdenum and zinc.

If appropriate, it is also possible for other additional components to be present, for example protective colloids, binders, adhesives, thickeners, thixotropic substances, penetrants, stabilizers, sequestrants, complexing agents. In general, the active ingredients can be combined with any solid or liquid additive commonly used for formulation purposes. In general, the compositions and formulations of the invention contain between 0.05% and 99% by weight, 0.01% and 98% by weight, preferably between 0.1% and 95% by weight, more preferably between 0.5% and 90% active ingredient, most preferably between 10 and 70 percent by weight. The active ingredients or compositions of the invention can be used as such or, depending on their respective physical and/or chemical properties, in the form of their formulations or the use forms prepared therefrom, such as aerosols, capsule suspensions, cold-fogging concentrates, warm-fogging concentrates, encapsulated granules, fine granules, flowable concentrates for the treatment of seed, ready-to-use solutions, dustable powders, emulsifiable concentrates, oil-in-water emulsions, water-in-oil emulsions, macrogranules, microgranules, oil-dispersible powders, oil-miscible flowable concentrates, oil-miscible liquids, foams, pastes, pesticide coated seed, suspension concentrates, suspoemulsion concentrates, soluble concentrates, suspensions, sprayable powders, soluble powders, dusts and granules, water-soluble granules or tablets, water-soluble powders for the treatment of seed, wettable powders, natural products and synthetic substances impregnated with active ingredient, and also microencapsulations in polymeric substances and in coating materials for seed, and also ULV cold-fogging and warm-fogging formulations.

The formulations mentioned can be produced in a manner known per se, for example by mixing the active ingredients with at least one customary extender, solvent or diluent, emulsifier, dispersant and/or binder or fixative, wetting agent, water repellent, optionally siccatives and UV stabilizers and optionally dyes and pigments, antifoams, preservatives, secondary thickeners, tackifiers, gibberellins and other processing auxiliaries.

The compositions of the invention include not only formulations which are already ready for use and can be deployed with a suitable apparatus onto the plant or the seed, but also commercial concentrates which have to be diluted with water prior to use.

The active ingredients of the invention may be present as such or in their (commercial standard) formulations, or else in the use forms prepared from these formulations as a mixture with other (known) active ingredients, such as insecticides, attractants, sterilants, bactericides, acaricides, nematicides, fungicides, growth regulators, herbicides, fertilizers, safeners or semiochemicals.

The inventive treatment of the plants and plant parts with the active ingredients or compositions is effected directly or by action on their surroundings, habitat or storage space by the customary treatment methods, for example by dipping, spraying, atomizing, irrigating, evaporating, dusting, fogging, broadcasting, foaming, painting, spreading-on, watering (drenching), drip irrigating and, in the case of propagation material, especially in the case of seeds, also by dry seed treatment, wet seed treatment, slurry treatment, incrustation, coating with one or more coats, etc. It is also possible to deploy the active ingredients by the ultra-low volume method or to inject the active ingredient preparation or the active ingredient itself into the soil.

One of the advantages of the present invention is that the particular systemic properties of the inventive active ingredients and compositions mean that treatment of the seed with these active ingredients and compositions protects not only the seed itself but also the resulting plants after emergence from phytopathogenic fungi. In this way, the immediate treatment of the crop at the time of sowing or shortly thereafter can be dispensed with.

It is likewise considered to be advantageous that the inventive active ingredients or compositions can especially also be used for transgenic seed, in which case the plant which grows from this seed is capable of expressing a protein which acts against pests. The treatment of such seed with the inventive active ingredients or compositions, merely through the expression of the protein, for example an insecticidal protein, can result in control of certain pests. Surprisingly, a further synergistic effect can be observed in this case, which additionally increases the effectiveness for protection against attack by pests.

The compositions of the invention are suitable for protection of seed of any plant variety which is used in agriculture, in the greenhouse, in forests or in horticulture and viticulture. In particular, this is the seed of cereals (such as wheat, barley, rye, triticale, sorghum/millet and oats), corn, cotton, soya beans, rice, potatoes, sunflower, bean, coffee, beet (for example sugar beet and fodder beet), peanut, oilseed rape, poppy, olive, coconut, cocoa, sugar cane, tobacco, vegetables (such as tomato, cucumbers, onions and lettuce), turf and ornamentals (see also below). The treatment of the seed of cereals (such as wheat, barley, rye, triticale and oats), corn and rice is of particular importance.

As also described below, the treatment of transgenic seed with the active ingredients or compositions of the invention is of particular significance. This relates to the seed of plants containing at least one heterologous gene which enables the expression of a polypeptide or protein having insecticidal properties. The heterologous gene in transgenic seed can originate, for example, from microorganisms of the species Bacillus, Rhizobium, Pseudomonas, Serratia, Trichoderma, Clavibacter, Glomus or Gliocladium. This heterologous gene preferably originates from Bacillus sp., in which case the gene product is effective against the European corn borer and/or the Western corn rootworm. The heterologous gene more preferably originates from Bacillus thuringiensis.

In the context of the present invention, the inventive composition is applied to the seed alone or in a suitable formulation. Preferably, the seed is treated in a state in which it is sufficiently stable for no damage to occur in the course of treatment. In general, the seed can be treated at any time between harvest and sowing. It is customary to use seed which has been separated from the plant and freed from cobs, shells, stalks, coats, hairs or the flesh of the fruits. For example, it is possible to use seed which has been harvested, cleaned and dried down to a moisture content of less than 15% by weight. Alternatively, it is also possible to use seed which, after drying, for example, has been treated with water and then dried again.

In general, when treating the seed, it has to be ensured that the amount of the composition of the invention and/or further additives applied to the seed is chosen such that the germination of the seed is not impaired and the plant which arises therefrom is not damaged. This has to be ensured particularly in the case of active ingredients which can exhibit phytotoxic effects at certain application rates.

The compositions of the invention can be applied directly, i.e. without containing any other components and without having been diluted. In general, it is preferable to apply the compositions to the seed in the form of a suitable formulation. Suitable formulations and methods for seed treatment are known to those skilled in the art and are described, for example, in the following documents: U.S. Pat. Nos. 4,272,417 A, 4,245,432 A, 4,808,430, 5,876,739, 2003/0176428 A1, WO 2002/080675 A1, WO 2002/028186 A2.

The active ingredients which can be used in accordance with the invention can be converted to the customary seed-dressing formulations, such as solutions, emulsions, suspensions, powders, foams, slurries or other coating compositions for seed, and also ULV formulations.

These formulations are produced in a known manner, by mixing the active ingredients with customary additives, for example customary extenders and solvents or diluents, dyes, wetting agents, dispersants, emulsifiers, antifoams, preservatives, secondary thickeners, adhesives, gibberellins, and also water.

Dyes which may be present in the seed-dressing formulations usable in accordance with the invention are all dyes which are customary for such purposes. It is possible to use either pigments, which are sparingly soluble in water, or dyes, which are soluble in water. Examples include the dyes known by the names Rhodamine B, C.I. Pigment Red 112 and C.I. Solvent Red 1.

Useful wetting agents which may be present in the seed-dressing formulations usable in accordance with the invention are all substances which promote wetting and which are customary for the formulation of agrochemically active ingredients. Alkyl naphthalenesulfonates, such as diisopropyl or diisobutyl naphthalenesulfonates, can be used with preference.

Suitable dispersants and/or emulsifiers which may be present in the seed-dressing formulations usable in accordance with the invention are all nonionic, anionic and cationic dispersants customary for the formulation of agrochemically active ingredients. Preference can be given to using nonionic or anionic dispersants or mixtures of nonionic or anionic dispersants. Suitable nonionic dispersants include especially ethylene oxide/propylene oxide block polymers, alkylphenol polyglycol ethers and tristryrylphenol polyglycol ethers, and the phosphated or sulfated derivatives thereof. Suitable anionic dispersants are especially lignosulfonates, polyacrylic acid salts and arylsulfonate-formaldehyde condensates.

Antifoams which may be present in the seed-dressing formulations usable in accordance with the invention are all foam-inhibiting substances customary for the formulation of agrochemically active ingredients. Silicone antifoams and magnesium stearate can be used with preference.

Preservatives which may be present in the seed-dressing formulations usable in accordance with the invention are all substances usable for such purposes in agrochemical compositions. Examples include dichlorophene and benzyl alcohol hemiformal.

Secondary thickeners which may be present in the seed-dressing formulations usable in accordance with the invention are all substances usable for such purposes in agrochemical compositions. Preferred examples include cellulose derivatives, acrylic acid derivatives, xanthan, modified clays and finely divided silica.

Useful stickers which may be present in the seed-dressing formulations usable in accordance with the invention are all customary binders usable in seed-dressing products. Preferred examples include polyvinylpyrrolidone, polyvinyl acetate, polyvinyl alcohol and tylose.

The seed-dressing formulations usable in accordance with the invention can be used, either directly or after previously having been diluted with water, for the treatment of a wide range of different seed, including the seed of transgenic plants. In this case, additional synergistic effects may also occur in interaction with the substances formed by expression.

For the treatment of seed with the seed-dressing formulations usable in accordance with the invention or with the preparations prepared therefrom by addition of water, useful equipment is all mixing units usable customarily for seed dressing. Specifically, the seed dressing procedure is to place the seed into a mixer, to add the particular desired amount of seed-dressing formulations, either as such or after prior dilution with water, and to mix them until the formulation is distributed homogeneously on the seed. If appropriate, this is followed by a drying operation.

The active ingredients of the invention, given good plant compatibility, favourable homeotherm toxicity and good environmental compatibility, are suitable for protection of plants and plant organs, for increasing harvest yields, and for improving the quality of the harvested crop. They can preferably be used as crop protection agents. They are active against normally sensitive and resistant species and also against all or specific stages of development.

The examples which follow illustrate the invention.

### A. CHEMICAL EXAMPLES

### Description of Chiral Separation

The compounds of the invention have been prepared by chiral supercritical liquid chromatography (SFC) separation of the corresponding racemates (Ia). The separation of the enantiomers was conducted using a Sepiatec SFC 100 instrument. By way of example, the conditions for the separations that led to the S enantiomers I-1, I-2, I-3, I-4, I-5 and I-6 are described below.

The enantiomeric purity of the separated enantiomers was determined by means of an analytical SFC system (Aquity UPC² from Waters). It is reported in the examples described below as enantiomeric excess ee. Optical rotation values were determined on a polarimeter from Krüss.

The S configuration of compounds I-1 and I-5 was ascertained by single-crystal x-ray structure analysis.

### Preparation of 2-chloro-N-(1,3,4-oxadiazol-2-yl)-3-[(S)-methylsulfinyl]-4-(trifluoromethyl)benzamide I-1

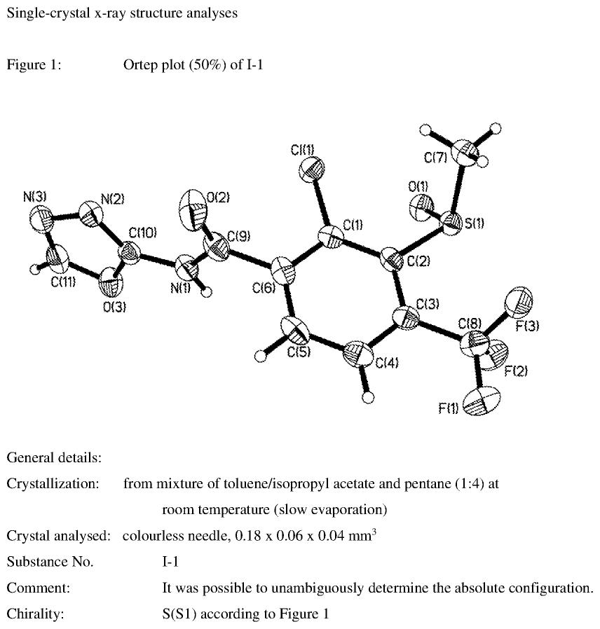

Racemate used: 2-chloro-N-(1,3,4-oxadiazol-2-yl)-3-[(rac)-methylsulfinyl]-4-(trifluoromethyl)benzamide (Ia-1)
Chiral column: Chiracel OX-H for SFC; 250×20 mm; 5 μm (Chiracel Technologies Europe)
Injection: 0.5 ml/injection of a solution of 850 mg of racemate 1a-1 in 19 ml of acetonitrile
Flow: 80 ml/min
Eluent: CO₂/MeOH=85/15
Peak: 4.30-4.80 min (S enantiomer I-1)
Analysis: S enantiomer I-1;
   SFC column: Chiracel OX-3 for SFC from Chiral Technologies Europe (0.3 cm×10 cm; 3 μm)
   Flow=2 ml/min.; CO₂/MeOH=90/10
   Retention time: 1.95 min.
   94% ee
   Optical rotation: −64° (MeOH)
   The absolute configuration was ascertained by single-crystal x-ray structure analysis. See FIG. 1.

### Preparation of 2-chloro-N-(1,3,4-oxadiazol-2-yl)-3-[(S)-methylsulfinyl]-4-(difluoromethyl)benzamide I-2

Racemate used: 2-chloro-N-(1,3,4-oxadiazol-2-yl)-3-[(rac)-methylsulfinyl]-4-(difluoromethyl)benzamide (Ia-2)
Chiral column: Lux® 5 μm Amylose-1; 250×21.2 mm (Phenomenex)
Injection: 0.5 ml/injection of a solution of 675 mg of racemate 1a-2 in 17 ml of acetonitrile
Flow: 80 ml/min
Eluent CO₂/MeOH=75/25
Peak: 1.80-2.80 min (S enantiomer I-2)
Analysis: S enantiomer I-2
   SFC column: Lux Amylose 1 from Phenomenex. (0.3 cm×150 mm; 3 μm)
   Flow=2 ml/min.; CO₂/MeOH=90/10
   Retention time: 1.91 min
   >99% ee
   Optical rotation: −194° (CHCl₃)
   The absolute configuration was assigned in analogy to I-1 and I-5.

### Preparation of 2-methyl-N-(1,3,4-oxadiazol-2-yl)-3-[(S)-methylsulfinyl]-4-(trifluoromethyl)benzamide I-3

Racemate used: 2-methyl-N-(1,3,4-oxadiazol-2-yl)-3-[(rac)-methylsulfinyl]-4-(trifluoromethyl)benzamide Ia-3
Chiral column: Chiracel OX-H for SFC; 250×20 mm; 5 μm (Chiracel Technologies Europe)
Injection: 0.3 ml/injection of a solution of 1.73 mg of racemate 1a-3 in 35 ml of acetonitrile
Flow: 80 ml/min
Eluent: CO₂/MeOH=85/15
Peak: 4.70-5.25 min (S enantiomer I-3)
Analysis: S enantiomer I-3
   SFC column: Chiracel OX-3 for SFC from Chiral Technologies Europe. (0.3 cm×10 cm; 3 μm)
   Flow=2 ml/min. (CO₂/MeOH=90/10)
   Retention time: 1.73 min
   >99% ee
   Optical rotation: −86° (MeOH)
   The absolute configuration was assigned in analogy to I-1 and I-5.

### Preparation of 2-chloro-N-(5-methyl-1,3,4-oxadiazol-2-yl)-3-[(S)-cyclopropylsulfinyl]-4-(trifluoromethyl)benzamide I-4

Racemate used: 2-chloro-N-(5-methyl-1,3,4-oxadiazol-2-yl)-3-[(rac)-cyclopropylsulfinyl]-4-(trifluoromethyl)benzamide 1a-4
Chiral column: Chiralpak AZH for SFC; 250×20 mm; 5 μm (Chiracel Technologies Europe)
Injection: 0.5 ml/injection of a solution of 139 mg of racemate 1a-4 in 5 ml of acetonitrile
Flow: 80 ml/min
Eluent CO₂/MeOH=85/15
Peak: 10.00-13.50 min (S enantiomer I-4)
Analysis: S enantiomer I-4
   SFC column: Chiracel AZ-3 for SFC from Chiral Technologies Europe (0.3 cm×10 cm; 3 μm)
   Flow=2 ml/min. (CO₂/MeOH=90/10)
   Retention time: 2.22 min.
   >99% ee
   Optical rotation: −43° (CHCl₃)
   The absolute configuration was assigned in analogy to I-1 and I-5.

### Preparation of 2-chloro-N-(5-methyl-1,3,4-oxadiazol-2-yl)-3-[(S)-methylsulfinyl]-4-(trifluoromethyl)benzamide I-5

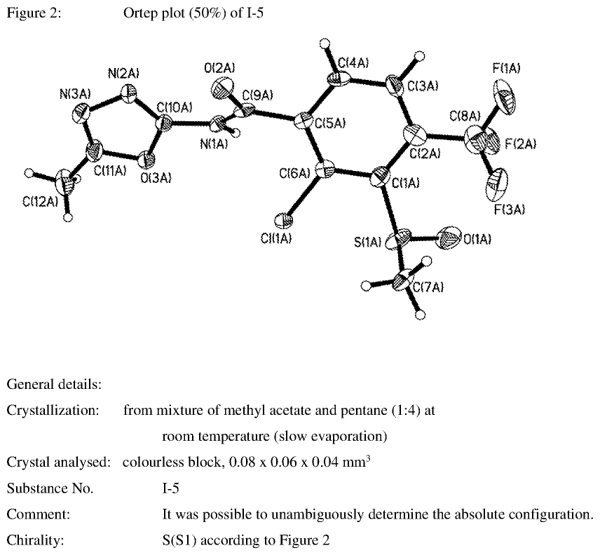

Racemate used: 2-chloro-N-(5-methyl-1,3,4-oxadiazol-2-yl)-3-[(rac)-methylsulfinyl]-4-(trifluoromethyl)benzamide 1a-5
Chiral column: Lux® 5 μm Amylose-1; 250×21.2 mm (Phenomenex)
Injection: 0.6 ml/injection of a solution of 100 mg of racemate 1a-5 in 2 ml of methanol
Flow: 80 ml/min
Eluent CO₂/MeOH=90/10
Peak: 4.60-6.37 min (S enantiomer I-5)
Analysis: S enantiomer I-5
   SFC column: Chiracel OX-3 for SFC from Chiral Technologies Europe (0.3 cm×10 cm; 3 μm)
   Flow=2 ml/min. (CO₂/MeOH=90/10)
   Retention time: 1.79 min
   >99% ee
   Optical rotation: −69° (MeOH)
   The absolute configuration was ascertained by single-crystal x-ray structure analysis. See FIG. 2.

### Preparation of 2-chloro-N-(5-methyl-1,3,4-oxadiazol-2-yl)-3-[(S)-methylsulfinyl]-4-(difluoromethyl)benzamide I-6

Racemate used: 2-chloro-N-(5-methyl-1,3,4-oxadiazol-2-yl)-3-[(rac)-methylsulfinyl]-4-(difluoromethyl)benzamide 1a-6
Chiral column: Chiracel OX-H for SFC; 250×20 mm; 5 μm (Chiracel Technologies Europe)
Injection: 0.5 ml/injection of a solution of 730 mg of racemate 1a-6 in 18 ml of acetonitrile
Flow: 80 ml/min
Eluent CO₂/MeOH=80/20
Peak 3.45-4.00 min; S enantiomer
Analysis: S enantiomer I-6
   SFC column: Chiracel OX-3 for SFC from Chiral Technologies Europe (0.3 cm×10 cm; 3 μm)
   Flow=2 ml/min. CO2/MeOH=90/10
   Retention time: 1.83 min.
   >99% ee
   Optical rotation: −178° (CHCl₃)
   The absolute configuration was assigned in analogy to I-1 and I-5.

The abbreviations used mean:

Me=methyl c-Pr=cyclopropyl

TABLE 1
Inventive compounds of the general formula (I) in which R, X, Z and R′ have the
definition given in Table 1.
(I)

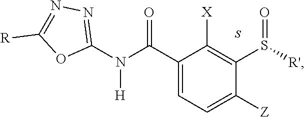

| No. | R | X | R' | Z | Optical rotation |
|---|---|---|---|---|---|
| I-1 | H | Cl | Me | CF₃ | (-)−65° (MeOH) |
| I-2 | H | Cl | Me | CHF₂ | (-)−194° (CHCl₃) |
| I-3 | H | Me | Me | CF₃ | (-)−86° (MeOH) |
| I-4 | Me | Cl | c-Pr | CF₃ | (-)−43° (CHCl₃) |
| I-5 | Me | Cl | Me | CF₃ | (-)−69° (MeOH) |
| I-6 | Me | Cl | Me | CHF₂ | (-)−178° (CHCl₃) |

### B. FORMULATION EXAMPLES

a) A dusting product is obtained by mixing 10 parts by weight of a compound of the formula (I) and/or salts thereof and 90 parts by weight of talc as an inert substance and comminuting the mixture in a hammer mill.
b) A readily water-dispersible, wettable powder is obtained by mixing 25 parts by weight of a compound of the formula (I) and/or salts thereof, 64 parts by weight of kaolin-containing quartz as an inert substance, 10 parts by weight of potassium lignosulfonate and 1 part by weight of sodium oleoylmethyltaurate as a wetting agent and dispersant, and grinding the mixture in a pinned-disk mill.
c) A readily water-dispersible dispersion concentrate is obtained by mixing 20 parts by weight of a compound of the formula (I) and/or salts thereof with 6 parts by weight of alkylphenol polyglycol ether (®Triton X 207), 3 parts by weight of isotridecanol polyglycol ether (8 EO) and 71 parts by weight of paraffinic mineral oil (boiling range for example about 255 to above 277 C), and grinding the mixture in a friction ball mill to a fineness of below 5 microns.
d) An emulsifiable concentrate is obtained from 15 parts by weight of a compound of the formula (I) and/or salts thereof, 75 parts by weight of cyclohexanone as a solvent and 10 parts by weight of ethoxylated nonylphenol as an emulsifier.
e) Water-dispersible granules are obtained by mixing
   75 parts by weight of a compound of the formula (I) and/or salts thereof,
   10 parts by weight of calcium lignosulfonate,
   5 parts by weight of sodium lauryl sulfate,
   3 parts by weight of polyvinyl alcohol and
   7 parts by weight of kaolin,
   grinding the mixture in a pinned-disk mill, and granulating the powder in a fluidized bed by spray application of water as a granulating liquid.
f) Water-dispersible granules are also obtained by homogenizing and precomminuting, in a colloid mill,
   25 parts by weight of a compound of the formula (I) and/or salts thereof,
   5 parts by weight of sodium 2,2′-dinaphthylmethane-6,6′-disulfonate
   2 parts by weight of sodium oleoylmethyltaurate,
   1 part by weight of polyvinyl alcohol,
   17 parts by weight of calcium carbonate and
   50 parts by weight of water,
   then grinding the mixture in a bead mill and atomizing and drying the resulting suspension in a spray tower by means of a one-phase nozzle.

### C. BIOLOGICAL EXAMPLES

The abbreviations used for the harmful plants mean:

| | | | |
|---|---|---|---|
| ABUTH | Abutilon theophrasti | ALOMY | Alopecurus myosuroides |
| AVEFA | Avena fatua | AMARE | Amaranthus retroflexus |
| CYPES | Cyperus esculentus | DIGSA | Digitaria sanguinalis |
| ECHCG | Echinochloa crus-galli | HORMU | Hordeum murinum |
| LOLMU | Lolium multiflorum | LOLRI | Lolium rigidum |
| MATIN | Matricaria inodora | PHBPU | Pharbitis purpurea |
| POLCO | Polygonum | SETVI | Setaria viridis |
| | convolvulus | | |
| STEME | Stellaria media | VERPE | Veronica persica |
| VIOTR | Viola tricolor | | |
| BRSNW | Brassica napus | GLXMA | Glycine max (soya) |
| | (winter oilseed rape) | | |
| ORYZA | Oryza sativa (rice) | TRZAS | Triticum aestiva |
| | | | (bread wheat) |
| ZEAMX | Zea mays (maize) | | |

### 1. Pre-Emergence Herbicidal Action Against Harmful Plants

Seeds of monocotyledonous and dicotyledonous weed plants and crop plants are laid out in sandy loam soil in wood-fibre pots and covered with soil. The compounds of the invention, formulated in the form of wettable powders (WP) or as emulsion concentrates (EC), are then applied to the surface of the covering soil in the form of an aqueous suspension or emulsion at a water application rate equivalent to 600 to 800 l/ha, with addition of 0.2% wetting agent. After the treatment, the pots are placed in a greenhouse and kept under good growth conditions for the trial plants. The damage to the test plants is scored visually after a test period of 3 weeks by comparison with untreated controls (herbicidal activity in percent (%): 100% activity=the plants have died, 0% activity=like control plants). Numerous compounds of the invention showed very good action against a multitude of important harmful plants. The tables below illustrate, in an illustrative manner, the post-emergence herbicidal action of the compounds of the invention, the herbicidal activity being stated in percent.

TABLE 1a
Pre-emergence action at 20 g/ha against ALOMY in %

| Example number | Dosage [g/ha] | ALOMY |
|---|---|---|
| I-4 | 20 | 80 |

TABLE 1b
Pre-emergence action at 80 g/ha against ALOMY in %

| Example number | Dosage [g/ha] | ALOMY |
|---|---|---|
| I-5 | 80 | 80 |
| I-1 | 80 | 80 |
| I-4 | 80 | 100 |
| I-6 | 80 | 100 |
| I-2 | 80 | 90 |

TABLE 2a
Pre-emergence action at 20 g/ha against AMARE in %

| Example number | Dosage [g/ha] | AMARE |
|---|---|---|
| I-5 | 20 | 90 |
| I-1 | 20 | 100 |
| I-3 | 20 | 100 |
| I-4 | 20 | 100 |
| I-6 | 20 | 90 |
| I-2 | 20 | 100 |

TABLE 2b
Pre-emergence action at 80 g/ha against AMARE in %

| Example number | Dosage [g/ha] | AMARE |
|---|---|---|
| I-5 | 80 | 100 |
| I-1 | 80 | 100 |
| I-3 | 80 | 100 |
| I-4 | 80 | 100 |
| I-6 | 80 | 100 |
| I-2 | 80 | 100 |

TABLE 3
Pre-emergence action at 80 g/ha against AVEFA in %

| Example number | Dosage [g/ha] | AVEFA |
|---|---|---|
| I-5 | 80 | 80 |
| I-1 | 80 | 80 |
| I-4 | 80 | 90 |
| I-6 | 80 | 100 |
| I-2 | 80 | 100 |

TABLE 4a
Pre-emergence action at 20 g/ha against DIGSA in %

| Example number | Dosage [g/ha] | DIGSA |
|---|---|---|
| I-3 | 20 | 100 |
| I-4 | 20 | 100 |
| I-6 | 20 | 100 |

TABLE 4b
Pre-emergence action at 80 g/ha against DIGSA in %

| Example number | Dosage [g/ha] | DIGSA |
|---|---|---|
| I-3 | 80 | 100 |
| I-4 | 80 | 100 |
| I-6 | 80 | 100 |

TABLE 5a
Pre-emergence action at 5 g/ha against ECHCG in %

| Example number | Dosage [g/ha] | ECHCG |
|---|---|---|
| I-1 | 5 | 90 |

TABLE 5b
Pre-emergence action at 20 g/ha against ECHCG in %

| Example number | Dosage [g/ha] | ECHCG |
|---|---|---|
| I-5 | 20 | 100 |
| I-1 | 20 | 100 |
| I-3 | 20 | 90 |
| I-6 | 20 | 90 |
| I-2 | 20 | 100 |

TABLE 5c
Pre-emergence action at 80 g/ha against ECHCG in %

| Example number | Dosage [g/ha] | ECHCG |
|---|---|---|
| I-5 | 80 | 100 |
| I-1 | 80 | 100 |
| I-3 | 80 | 100 |
| I-4 | 80 | 100 |
| I-6 | 80 | 100 |
| I-2 | 80 | 100 |

TABLE 6
Pre-emergence action at 80 g/ha against LOLRI in %

| Example number | Dosage [g/ha] | LOLRI |
|---|---|---|
| I-4 | 80 | 80 |
| I-2 | 80 | 90 |

TABLE 7a
Pre-emergence action at 20 g/ha against MATIN in %

| Example number | Dosage [g/ha] | MATIN |
|---|---|---|
| I-5 | 20 | 80 |
| I-1 | 20 | 100 |
| I-4 | 20 | 80 |
| I-6 | 20 | 90 |
| I-2 | 20 | 100 |

TABLE 7b
Pre-emergence action at 80 g/ha against MATIN in %

| Example number | Dosage [g/ha] | MATIN |
|---|---|---|
| I-5 | 80 | 100 |
| I-1 | 80 | 100 |
| I-3 | 80 | 90 |
| I-4 | 80 | 100 |
| I-6 | 80 | 90 |
| I-2 | 80 | 100 |

TABLE 8
Pre-emergence action at 80 g/ha against PHBPU in %

| Example number | Dosage [g/ha] | PHBPU |
|---|---|---|
| I-5 | 80 | 100 |
| I-1 | 80 | 100 |

TABLE 9
Pre-emergence action at 80 g/ha against POLCO in %

| Example number | Dosage [g/ha] | POLCO |
|---|---|---|
| I-1 | 80 | 80 |
| I-4 | 80 | 80 |
| I-2 | 80 | 90 |

TABLE 10a
Pre-emergence action at 5 g/ha against SETVI in %

| Example number | Dosage [g/ha] | SETVI |
|---|---|---|
| I-5 | 5 | 80 |

TABLE 10b
Pre-emergence action at 20 g/ha against SETVI in %

| Example number | Dosage [g/ha] | SETVI |
|---|---|---|
| I-5 | 20 | 100 |
| I-1 | 20 | 80 |
| I-4 | 20 | 100 |
| I-6 | 20 | 90 |
| I-2 | 20 | 100 |

TABLE 10c
Pre-emergence action at 80 g/ha against SETVI in %

| Example number | Dosage [g/ha] | SETVI |
|---|---|---|
| I-5 | 80 | 100 |
| I-1 | 80 | 100 |
| I-3 | 80 | 90 |
| I-4 | 80 | 100 |
| I-6 | 80 | 100 |
| I-2 | 80 | 100 |

TABLE 11a
Pre-emergence action at 20 g/ha against STEME in %

| Example number | Dosage [g/ha] | STEME |
|---|---|---|
| I-3 | 20 | 80 |

TABLE 11b
Pre-emergence action at 80 g/ha against STEME in %

| Example number | Dosage [g/ha] | STEME |
|---|---|---|
| I-3 | 80 | 100 |

TABLE 12a
Pre-emergence action at 20 g/ha against VERPE in %

| Example number | Dosage [g/ha] | VERPE |
|---|---|---|
| I-5 | 20 | 80 |
| I-6 | 20 | 90 |
| I-2 | 20 | 80 |

TABLE 12b
Pre-emergence action at 80 g/ha against VERPE in %

| Example number | Dosage [g/ha] | VERPE |
|---|---|---|
| I-5 | 80 | 100 |
| I-4 | 80 | 100 |
| I-6 | 80 | 100 |
| I-2 | 80 | 80 |

TABLE 13a
Pre-emergence action at 20 g/ha against VIOTR in %

| Example number | Dosage [g/ha] | VIOTR |
|---|---|---|
| I-1 | 20 | 100 |
| I-3 | 20 | 90 |
| I-4 | 20 | 100 |
| I-6 | 20 | 100 |
| I-2 | 20 | 100 |

TABLE 13b
Pre-emergence action at 80 g/ha against VIOTR in %

| Example number | Dosage [g/ha] | VIOTR |
|---|---|---|
| I-5 | 80 | 100 |
| I-1 | 80 | 100 |
| I-3 | 80 | 100 |
| I-4 | 80 | 100 |
| I-6 | 80 | 100 |
| I-2 | 80 | 100 |

### 2. Post-Emergence Herbicidal Action Against Harmful Plants

Seeds of monocotyledonous and dicotyledonous weed and crop plants are laid out in sandy loam soil in wood-fibre pots, covered with soil and cultivated in a greenhouse under good growth conditions. 2 to 3 weeks after sowing, the test plants are treated at the one-leaf stage. The compounds of the invention, formulated in the form of wettable powders (WP) or as emulsion concentrates (EC), are then sprayed onto the green parts of the plants in the form of an aqueous suspension or emulsion at a water application rate equating to 600 to 800 l/ha, with addition of 0.2% wetting agent. After the test plants have been left to stand in the greenhouse under optimal growth conditions for about 3 weeks, the action of the preparations is assessed visually in comparison to untreated controls (herbicidal action in percent (%): 100% activity=the plants have died, 0% activity=like control plants). Numerous compounds of the invention showed very good action against a multitude of important harmful plants. The tables below illustrate, in an illustrative manner, the post-emergence herbicidal action of the compounds of the invention, the herbicidal activity being stated in percent.

TABLE 14a
Post-emergence action at 1.25 g/ha against AMARE in %

| Example number | Dosage [g/ha] | AMARE |
|---|---|---|
| I-1 | 1.25 | 100 |

TABLE 14b
Post-emergence action at 5 g/ha against AMARE in %

| Example number | Dosage [g/ha] | AMARE |
|---|---|---|
| I-5 | 5 | 100 |
| I-1 | 5 | 100 |
| I-3 | 5 | 90 |
| I-4 | 5 | 100 |
| I-6 | 5 | 90 |
| I-2 | 5 | 100 |

TABLE 14c
Post-emergence action at 20 g/ha against AMARE in %

| Example number | Dosage [g/ha] | AMARE |
|---|---|---|
| I-5 | 20 | 100 |
| I-1 | 20 | 100 |
| I-3 | 20 | 90 |
| I-4 | 20 | 100 |
| I-6 | 20 | 100 |
| I-2 | 20 | 100 |

TABLE 15
Post-emergence action at 20 g/ha against AVEFA in %

| Example number | Dosage [g/ha] | AVEFA |
|---|---|---|
| I-1 | 20 | 80 |

TABLE 16a
Post-emergence action at 5 g/ha against DIGSA in %

| Example number | Dosage [g/ha] | DIGSA |
|---|---|---|
| I-3 | 5 | 90 |
| I-4 | 5 | 90 |
| I-6 | 5 | 90 |

TABLE 16b
Post-emergence action at 20 g/ha against DIGSA in %

| Example number | Dosage [g/ha] | DIGSA |
|---|---|---|
| I-3 | 20 | 90 |
| I-4 | 20 | 100 |
| I-6 | 20 | 90 |

TABLE 17a
Post-emergence action at 5 g/ha against ECHCG in %

| Example number | Dosage [g/ha] | ECHCG |
|---|---|---|
| I-5 | 5 | 90 |
| I-1 | 5 | 100 |
| I-3 | 5 | 80 |
| I-2 | 5 | 90 |

TABLE 17b
Post-emergence action at 20 g/ha against ECHCG in %

| Example number | Dosage [g/ha] | ECHCG |
|---|---|---|
| I-5 | 20 | 100 |
| I-1 | 20 | 100 |
| I-3 | 20 | 90 |
| I-4 | 20 | 90 |
| I-6 | 20 | 100 |
| I-2 | 20 | 90 |

TABLE 18a
Post-emergence action at 5 g/ha against MATIN in %

| Example number | Dosage [g/ha] | MATIN |
|---|---|---|
| I-5 | 5 | 80 |
| I-1 | 5 | 80 |
| I-2 | 5 | 90 |

TABLE 18b
Post-emergence action at 20 g/ha against MATIN in %

| Example number | Dosage [g/ha] | MATIN |
|---|---|---|
| I-5 | 20 | 90 |
| I-1 | 20 | 90 |
| I-3 | 20 | 80 |
| I-4 | 20 | 80 |
| I-2 | 20 | 90 |

TABLE 19a
Post-emergence action at 5 g/ha against PHBPU in %

| Example number | Dosage [g/ha] | PHBPU |
|---|---|---|
| I-1 | 5 | 100 |
| I-3 | 5 | 80 |

TABLE 19b
Post-emergence action at 20 g/ha against PHBPU in %

| Example number | Dosage [g/ha] | PHBPU |
|---|---|---|
| I-5 | 20 | 90 |
| I-1 | 20 | 100 |
| I-3 | 20 | 80 |
| I-6 | 20 | 90 |
| I-2 | 20 | 90 |

TABLE 20
Post-emergence action at 20 g/ha against POLCO in %

| Example number | Dosage [g/ha] | POLCO |
|---|---|---|
| I-1 | 20 | 80 |
| I-2 | 20 | 80 |

TABLE 21a
Post-emergence action at 5 g/ha against SETVI in %

| Example number | Dosage [g/ha] | SETVI |
|---|---|---|
| I-5 | 5 | 80 |
| I-1 | 5 | 80 |
| I-4 | 5 | 100 |
| I-6 | 5 | 100 |
| I-2 | 5 | 90 |

TABLE 21b
Post-emergence action at 20 g/ha against SETVI in %

| Example number | Dosage [g/ha] | SETVI |
|---|---|---|
| I-5 | 20 | 100 |
| I-1 | 20 | 100 |
| I-3 | 20 | 90 |
| I-4 | 20 | 100 |
| I-6 | 20 | 100 |
| I-2 | 20 | 90 |

TABLE 22
Post-emergence action at 20 g/ha against STEME in %

| Example number | Dosage [g/ha] | STEME |
|---|---|---|
| I-3 | 20 | 80 |

TABLE 23a
Post-emergence action at 5 g/ha against VERPE in %

| Example number | Dosage [g/ha] | VERPE |
|---|---|---|
| I-6 | 5 | 90 |
| I-2 | 5 | 90 |

TABLE 23b
Post-emergence action at 20 g/ha against VERPE in %

| Example number | Dosage [g/ha] | VERPE |
|---|---|---|
| I-5 | 20 | 90 |
| I-1 | 20 | 90 |
| I-6 | 20 | 90 |
| I-2 | 20 | 100 |

TABLE 24a
Post-emergence action at 1.25 g/ha against VIOTR in %

| Example number | Dosage [g/ha] | VIOTR |
|---|---|---|
| I-1 | 1.25 | 90 |

TABLE 24b
Post-emergence action at 5 g/ha against VIOTR in %

| Example number | Dosage [g/ha] | VIOTR |
|---|---|---|
| I-5 | 5 | 100 |
| I-1 | 5 | 100 |
| I-6 | 5 | 80 |
| I-2 | 5 | 90 |

TABLE 24c
Post-emergence action at 20 g/ha against VIOTR in %

| Example number | Dosage [g/ha] | VIOTR |
|---|---|---|
| I-5 | 20 | 100 |
| I-1 | 20 | 100 |
| I-3 | 20 | 90 |
| I-6 | 20 | 100 |
| I-2 | 20 | 100 |

### COMPARATIVE EXPERIMENTS

In the experiments that follow, the herbicidal action of the compounds of the invention and that of the corresponding known racemic compounds closest from WO2012126932 and WO2018202535 were compared under the above-specified conditions by the pre-emergence and post-emergence method. The example numbers given in the tables relate to the compounds disclosed in the respective documents.

TABLE 25
Herbicidal pre-emergence action/damage to crop plants

| | Dosage | Damage to |
|---|---|---|
| Example No.: | (g a.i./ha) | ZEAMX |
| I-1, inventive | 320 | 0 |
| 1-360, from WO2012126932 | 320 | 20 |

| | Dosage | Damage to | |
|---|---|---|---|
| Example No.: | (g a.i./ha) | ORYZA | TRZAS |
| I-1, inventive | 80 | 80 | 30 |
| 1-360, from WO2012126932 | 80 | 100 | 80 |

| | Dosage | Damage to |
|---|---|---|
| Example No.: | (g a.i./ha) | TRZAS |
| I-2, inventive | 20 | 20 |
| Racemate, known from WO2018202535 | 20 | 70 |

| | Dosage | Damage to | |
|---|---|---|---|
| Example No.: | (g a.i./ha) | GLXMA | TRZAS |
| I-2, inventive | 80 | 30 | 70 |
| Racemate, known from | 80 | 80 | 100 |
| WO2018202535 | | | |

| | Dosage | Damage to | | |
|---|---|---|---|---|
| Example No.: | (g a.i./ha) | GLXMA | ZEAMX | TRZAS |
| I-3, inventive | 80 | 50 | 0 | 30 |
| 1-144 from | 80 | 80 | 30 | 90 |
| WO2012126932 | | | | |
| | Dosage | Damage to | | |
| Example No.: | (g a.i./ha) | BRSNW | ZEAMX | TRZAS |
| I-3, inventive | 20 | 70 | 0 | 0 |
| 1-144 from | 20 | 100 | 20 | 50 |
| WO2012126932 | | | | |

| | |
|---|---|
| Dosage | Damage to |

| Example No.: | (g a.i./ha) | GLXMA | ZEAMX |
|---|---|---|---|
| I-4, inventive | 80 | 80 | 50 |
| Racemate known from WO2012126932 | 80 | 100 | 70 |

| | | Damage to |
|---|---|---|
| Example No.: | Dosage (g a.i./ha) | GLXMA |
| I-4, inventive | 20 | 40 |
| Racemate known from WO2012126932 | 20 | 60 |

| | Dosage | Herbicidal action against | | |
|---|---|---|---|---|
| Example No.: | (g a.i./ha) | ALOMY | AVEFA | LOLRI |
| I-4, inventive | 20 | 80 | 30 | 50 |
| Racemate known from | 20 | 0 | 0 | 0 |
| WO2012126932 | | | | |

| | Dosage | Herbicidal action against |
|---|---|---|
| Example No.: | (g a.i./ha) | ZEAMX |
| I-5, inventive | 320 | 40 |
| 2-360, from WO2012126932 | 320 | 60 |

| | Dosage | Herbicidal action against | |
|---|---|---|---|
| Example No.: | (g a.i./ha) | STEME | POLCO |
| I-5, inventive | 320 | 100 | 80 |
| 2-360, from WO2012126932 | 320 | 70 | 20 |
| | Dosage | Herbicidal action against | |
| Example No.: | (g a.i./ha) | STEME | VERPE |
| I-5, inventive | 80 | 100 | 100 |
| 2-360, from WO2012126932 | 80 | 70 | 10 |

| | Dosage | Herbicidal action against | | | |
|---|---|---|---|---|---|
| Example No.: | (g a.i./ha) | ALOMY | AVEFA | CYPES | MATIN |
| I-5, inventive | 20 | 60 | 90 | 90 | 90 |
| 2-360, from | 20 | 30 | 0 | 70 | 70 |
| WO2012126932 | | | | | |

| | Dosage | Herbicidal action against | | |
|---|---|---|---|---|
| Example No.: | (g a.i./ha) | SETVI | STEME | VERPE |
| I-5, inventive | 20 | 100 | 100 | 30 |
| 2-360, from | 20 | 50 | 0 | 0 |
| WO2012126932 | | | | |

| | Dosage | Damage to |
|---|---|---|
| Example No.: | (g a.i./ha) | ZEAMX |
| I-6, inventive | 80 | 20 |
| 7-48 from WO2018202535 | 80 | 50 |
| | Dosage | Damage to |
| Example No.: | (g a.i./ha) | BRSNW |
| I-6, inventive | 20 | 40 |
| 7-48 from WO2018202535 | 20 | 80 |

TABLE 26
Herbizide Wirkung im Nachauflauf/Schädigung von Kulturpflanzen

| | Dosage | Damage to | | |
|---|---|---|---|---|
| Example No.: | (g a.i./ha) | ORYZA | ZEAMX | TRZAS |
| I-1, inventive | 80 | 70 | 0 | 60 |
| 1-360, from | 80 | 100 | 20 | 100 |
| WO2012126932 | | | | |

| | Dosage | Damage to |
|---|---|---|
| Example No.: | (g a.i./ha) | ZEAMX |
| I-1, inventive | 80 | 0 |
| 1-360, from WO2012126932 | 80 | 20 |

| | Dosage | Damage to |
|---|---|---|
| Example No.: | (g a.i./ha) | ORYZA |
| I-2, inventive | 5 | 0 |
| Racemate, known from WO2018202535 | 5 | 60 |

I-2, post-emergence action

| | Dosage | Herbicidal action against |
|---|---|---|
| Example No.: | (g a.i./ha) | ECHCG |
| I-2, inventive | 5 | 90 |
| Racemate, known from WO2018202535 | 5 | 70 |

| | Dosage | Damage to | |
|---|---|---|---|
| Example No.: | (g a.i./ha) | ORYZA | TRZAS |
| I-3, inventive | 5 | 30 | 20 |
| 1-144 from WO2012126932 | 5 | 60 | 40 |

| | Dosage | Damage to |
|---|---|---|
| Example No.: | (g a.i./ha) | TRZAS |
| I-3, inventive | 20 | 30 |
| 1-144 from WO2012126932 | 20 | 50 |

| | Dosage | Damage to | | |
|---|---|---|---|---|
| Example No.: | (g a.i./ha) | BRSNW | ZEAMX | TRZAS |
| I-4, inventive | 20 | 80 | 0 | 20 |
| Racemate, known from | 20 | 100 | 20 | 40 |
| WO2012126932 | | | | |

| | Dosage | Herbicidal action against | |
|---|---|---|---|
| Example No.: | (g a.i./ha) | LOLRI | DIGSA |
| I-4, inventive | 20 | 60 | 100 |
| Racemate, known from WO2012126932 | 20 | 30 | 80 |

| | Dosage | Herbicidal action against |
|---|---|---|
| Example No.: | (g a.i./ha) | VERPE |
| I-4, inventive | 5 | 60 |
| Racemate, known from WO2012126932 | 5 | 40 |

| | Dosage | Damage to |
|---|---|---|
| Example No.: | (g a.i./ha) | ZEAMX |
| I-5, inventive | 20 | 0 |
| 2-360, from WO2012126932 | 20 | 20 |

| | Dosage | Herbicidal action against | | |
|---|---|---|---|---|
| Example No.: | (g a.i./ha) | SETVI | STEME | VERPE |
| I-5, inventive | 5 | 100 | 80 | 40 |
| 2-360, from | 5 | 80 | 0 | 20 |
| WO2012126932 | | | | |

| | Dosage | Herbicidal action against |
|---|---|---|
| Example No.: | (g a.i./ha) | CYPES |
| I-5, inventive | 20 | 80 |
| 2-360, from WO2012126932 | 20 | 50 |

I-6 PO selectivity

| | Dosage | Damage to | |
|---|---|---|---|
| Example No.: | (g a.i./ha) | GLXMA | TRZAS |
| I-6, inventive | 5 | 40 | 20 |
| 7-48 from WO2018202535 | 5 | 60 | 40 |

| | Dosage | Herbicidal action against | |
|---|---|---|---|
| Example No.: | (g a.i./ha) | ECHCG | VIOTR |
| I-6, inventive | 20 | 100 | 100 |
| 7-48 from WO2018202535 | 20 | 80 | 80 |
| | Dosage | Herbicidal action against | |
| Example No.: | (g a.i./ha) | SETVI | VIOTR |
| I-6, inventive | 5 | 100 | 80 |
| 7-48 from WO2018202535 | 5 | 80 | 60 |

1. An N-(1,3,4-Oxadiazole-2-yl)phenyl carboxamide or salt thereof, of absolute configuration specified in formula (I) 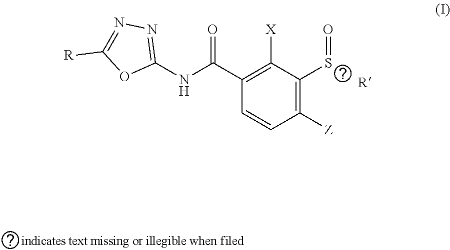
in which substituents are defined as follows:
R is hydrogen or methyl,
X is Cl or methyl,
R′ is methyl or c-Pr,
Z is CF₃ or CHF₂.

2. The N-(1,3,4-Oxadiazole-2-yl)phenyl carboxamide or salt according to claim 1 comprising one or more of the following:

| No. | R | X | R′ | Z |
|---|---|---|---|---|
| I-1 | H | Cl | Me | CF₃ |
| I-2 | H | Cl | Me | CHF₂ |
| I-3 | H | Me | Me | CF₃ |
| I-4 | Me | Cl | c-Pr | CF₃ |
| I-5 | Me | Cl | Me | CF₃ |
| I-6 | Me | Cl | Me | CHF₂ |
| I-7 | H | Me | Me | CHF₂ |
| I-8 | H | Me | c-Pr | CF₃ |
| I-9 | H | Me | c-Pr | CHF₂ |
| I-10 | H | Cl | c-Pr | CF₃ |
| I-11 | H | Cl | c-Pr | CHF₂ |
| I-12 | Me | Cl | c-Pr | CHF₂ |
| I-13 | Me | Me | Me | CF₃ |
| I-14 | Me | Me | Me | CHF₂ |
| I-15 | Me | Me | c-Pr | CF₃ |
| I-16 | Me | Me | c-Pr | CHF₂ |

3. The N-(1,3,4-Oxadiazole-2-yl)phenyl carboxamide or salt according to claim 1 comprising one or more of the following:

| No. | R | X | R′ | Z |
|---|---|---|---|---|
| I-1 | H | Cl | Me | CF₃ |
| I-2 | H | Cl | Me | CHF₂ |
| I-3 | H | Me | Me | CF₃ |
| I-4 | Me | Cl | c-Pr | CF₃ |
| I-5 | Me | Cl | Me | CF₃ |

4. The N-(1,3,4-Oxadiazole-2-yl)phenyl carboxamide or salt according to claim 1 comprising one or more of the following:

| No. | R | X | R′ | Z |
|---|---|---|---|---|
| I-4 | Me | Cl | c-Pr | CF₃ |
| I-5 | Me | Cl | Me | CF₃ |

5. The N-(1,3,4-Oxadiazole-2-yl)phenyl carboxamide or salt according to claim 1 with an enantiomeric excess (ee) of at least 94%.

6. The N-(1,3,4-Oxadiazole-2-yl)phenyl carboxamide or salt according to claim 1 with an enantiomeric excess (ee) of at least 99%.

7. An Herbicidal composition comprising at least one compound according to claim 1, mixed with one or more formulation auxiliaries.

8. The Herbicidal composition according to claim 6, comprising at least one further pesticidally active substance from the group consisting of insecticides, acaricides, herbicides, fungicides, safeners, and growth regulators.

9. A Method of controlling one or more unwanted plants, comprising applying an effective amount of at least one compound according to claim 1 to plants or a site of unwanted vegetation.

10. A compound according to claim 1 for controlling one or more unwanted plants.

11. The compound according to claim 10, wherein the compound is used for controlling unwanted plants in one or more crops of one or more useful plants.

12. The compound according to claim 11, wherein the useful plants are transgenic useful plants.
